(19) United States
(12) Patent Application Publication
(10) Pub. No.: US 2025/0017771 A1
(43) Pub. Date: Jan. 16, 2025

(54) Multiple Curved Segments in a Cannula for Cannulation of the Mammalian Eye

(71) Applicant: eyeFlow, Inc., Mission Viejo, CA (US)

(72) Inventors: Douglas CALI, Mission Viejo, CA (US); Marcus SOUZA, Costa Mesa, CA (US)

(21) Appl. No.: 18/773,443

(22) Filed: Jul. 15, 2024

Related U.S. Application Data
(60) Provisional application No. 63/526,921, filed on Jul. 14, 2023.

(51) Int. Cl. A61F 9/007

(52) U.S. Cl. CPC A61F 9/007

(57) ABSTRACT

A multi curved cannula comprising a proximal curved segment having a proximal end and a distal end, and a distal curved segment having a proximal end and a distal end, wherein the proximal end of the distal curved segment is adjacent the distal end of the proximal curved segment to form the multiple curved cannula.

### CLAIM OF PRIORITY

This application claims the benefit of U.S. Provisional Patent Application No. 63/526,921, filed Jul. 14, 2023, entitled “MULTIPLE CURVED SEGMENTS IN A CANNULA FOR IMPROVED CANNULATION OF THE MAMMALIAN EYE”, the disclosure of which is incorporated by reference herein in its entirety.

### COPYRIGHT NOTICE

This document contains material that is subject to copyright protection. The copyright owner has no objection to the facsimile reproduction by anyone of the document as it appears in the Patent and Trademark Office records, but otherwise reserves all copyright rights whatsoever.

### TECHNICAL FIELD

Embodiments of the invention relate to a method and apparatus for insertion of a medical device into a mammalian eye.

### BACKGROUND

Ophthalmological surgery on the mammalian eye that involves inserting a medical device into the eye often involves inserting a cannula, defined as a hollow needle, into the tissues of the eye or the anatomical spaces of the eye. The medical device is then inserted into the eye via the hollow portion, or lumen, of the cannula. Insertion of the cannula allows for precise delivery of the medical device through the lumen, i.e., an inner hollow portion, or interior tube portion, of the cannula, into the tissues or anatomical spaces of the eye. The dimensions and shape of the cannula play a critical role in successful delivery of the medical device into the tissues or anatomical spaces of the eye. A simple straight cannula shape often can lead to very complicated manipulation of the cannula for successful delivery of the medical device into the tissues or anatomical spaces of the eye. Similarly, a simply curved cannula, defined as a cannula that has a single curved segment, may also involve complicated manipulation of the cannula to successfully deliver the medical device into the tissues or anatomical spaces of the eye. Inserting a straight cannula or a simply curved cannula may also require multiple incisions in the eye during surgical procedures on the eye. What is needed is a cannula design that simplifies manipulation of the cannula for successful delivery of a medical device into the tissues or anatomical spaces of the eye and that reduces incisions to the eye during surgical procedures on the eye, ideally reducing incisions to the eye to a single incision to the eye during surgical procedures involving the eye.

### SUMMARY

A multiple curved cannula minimizes the number of incisions to the eye and other structures of the eye during eye surgery and enables precise positioning of the cannula tip inside the eye during eye surgery. Embodiments of the invention comprise a multiple curved cannula that has two (2) or more curved segments. The curved segments of the cannula allow for the cannula distal tip to be accurately positioned in the eye, for example, to enable accurate placement of a medical device into the eye, and/or to minimize the number of incisions to the eye and other structures of the eye during eye surgery.

### BRIEF DESCRIPTION OF THE DRAWINGS

Embodiments are illustrated by way of example, and not by way of limitation, and will be more fully understood with reference to the following written description when considered in connection with the figures, in which like reference numbers refer to like parts, and in which:

FIG. 1 illustrates a multiple curved cannula, according to embodiments of the invention; and

FIG. 2 illustrates geometric segments of a multiple curved cannula, according to embodiments of the invention.

### DETAILED DESCRIPTION

Embodiments of the invention comprise a multiple curved cannula having 2 or more curved segments or portions for the delivery of a medical device into a mammalian eye. The multiple curved cannula may be manufactured from a variety of materials including but not limited to metal, plastic, or glass, or a combination thereof. In one embodiment, the segments are manufactured as separate curved components that are then assembled into the multiple curved cannula. In another embodiment, the multiple curved cannula is manufactured as a single component that has multiple curved segments or portions.

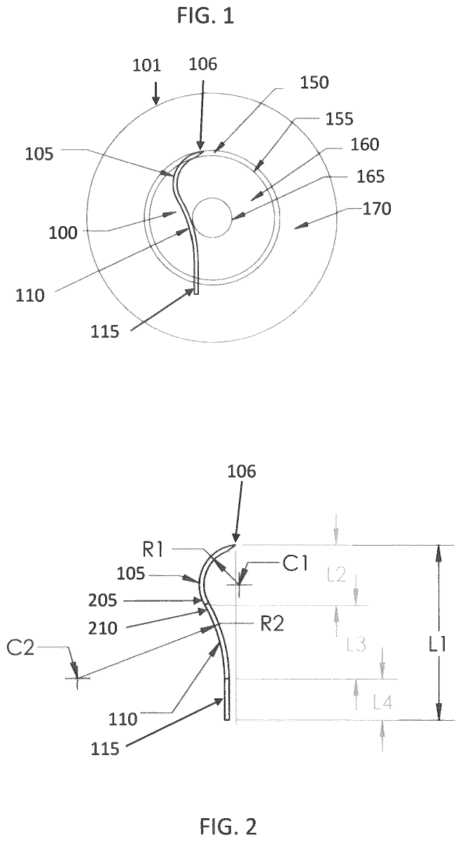

FIG. 1 illustrates a multiple curved cannula 100, according to embodiments of the invention, comprising two curved segments including distal curved segment 105 and proximal curved segment 110. In the disclosed embodiments, “distal” and “proximal” are meant to refer to the respective locations of segments in relation to the location of the inserter handle of the cannula. Similarly, a “proximal end” and a “distal end” of a segment is meant to refer to the respective orientation and location of the ends of a given segment in relation to the location of the inserter handle of the cannula.

With reference to FIG. 2, in the illustrated embodiment, the distal curved segment 105 and the proximal curved segment 110 are adjacent (i.e., there may or may not be contact between the two segments, but there is an absence of another curved segment therebetween), adjoin one another (i.e., the segments are in complete contact with each other), or are contiguous with one another (i.e., a proximal end 205 of the distal curved segment 105 is in contact with all or most of a distal end 210 of the proximal curved segment 110. In another embodiment (not illustrated), a linear or straight segment may connect the distal curved segment 105 to the proximal curved segment 110, even while the overall configuration of the cannula shape still essentially involves two curved segments.

Multiple curved cannula 100 further comprises straight segment 115. A distal end of straight segment 115 connects to a proximal end of proximal curved segment 110. A proximal end of straight segment 115 connects or leads to a grip or inserter handle of the cannula 100, i.e., the straight segment 115 connects directly to the inserter handle or connects indirectly to the inserter handle via another one or more components such as another one or more straight segments, another one or more curved segments, or a combination thereof (not shown).

FIG. 1 depicts a mammalian eye 101, including Schlemm's canal 150, trabecular meshwork 155, iris 160, pupil 165 and schlera 170. Multiple curved cannula 100 is illustrated in FIG. 1 placed into the anterior chamber of the eye 101 such that the cannula distal tip 106 is positioned in or adjacent to Schlemm's canal 150 with the axial direction of the distal lumen of the cannula approximately co linear to the tangent of the direction of Schlemm's canal at the same location.

While FIG. 1 depicts multiple curved cannula 100 placed into the anterior chamber of the eye, it is appreciated that multiple curved cannula may be inserted and then positioned into various locations of the eye, depending on the type of procedure being performed. For example, it is contemplated the multiple curved cannula according to embodiments of the invention may be used for one or more of the following purposes: to insert a medical device into the anterior segment of the eye; to insert a medical device into a posterior segment of the eye; to insert a medical device into the Schlemm's canal anatomy of the eye; to insert a medical device into the Trabecular Meshwork anatomy of the eye; to insert a medical device into the Supraciliary Space of the anatomy of the eye; to insert a stent into the Schlemm's canal anatomy of the eye; to insert a tube into the Schlemm's canal anatomy of the eye; to insert a catheter into the Schlemm's canal anatomy of the eye; to insert a wire into the Schlemm's canal anatomy of the eye; to insert a polymer filament into the Schlemm's canal anatomy of the eye; to insert a polymer suture into the Schlemm's canal anatomy of the eye; to insert a drug eluting medical device into the Schlemm's canal anatomy of the eye; to insert a liquid based medical device into the Schlemm's canal anatomy of the eye; to insert a viscoelastic medical device into the Schlemm's canal anatomy of the eye; to insert a polymer gel medical device into the Schlemm's canal anatomy of the eye; to insert a drug eluting medical device into the Trabecular Meshwork of the eye; and to insert a drug eluting medical device into the Supraciliary Space of the eye.

FIG. 2 illustrates geometric segments of a multiple curved cannula 100, according to embodiments of the invention. The multiple curved cannula 100 comprises two curved segments, a distal curved segment 105 and a proximal curved segment 110. According to an embodiment, the center of the radius R1 of curvature C1 for distal curved segment 105 is at a different location than the location of the center of radius R2 of curvature C2 for proximal curved segment 110. In the illustrated embodiment, the two curved segments are adjacent. However, it is appreciated that an intermediate segment, e.g., an intermediate linear or straight segment may couple the two curved segments. It is further appreciated that the length of the intermediate segment may be relatively shorter in length compared to the length of either one or both curved segments. It is also appreciated that the lengths of the curved segments may be the same or differ with respect to each other, i.e., the distal curved segment 105 may the same length as the proximal curved segment 110 or may be shorter or longer in length than the proximal curved segment 110.

In the embodiments illustrated in FIGS. 1 and 2, distal curved segment 105 defines an arc, i.e., a part or segment of the circumference of a first circle having a first circumference, and proximal curved segment 110 defines another arc of the circumference of a second circle having a second circumference.

In the illustrated embodiments, the circumference of the first circle defined by distal curved segment 105 is less than the circumference of the second circle defined by the proximal curved segment 110. In one embodiment, the circumference of the first circle approximates the circumference of Schlemm's canal (i.e., approximately 36-40 mm, which is the generally accepted circumference of Schlemm's canal) and may be slightly greater or less than the circumference of Schlemm's canal in other embodiments.

The circumference of distal curved segment 105, according to one embodiment, directly corresponds to, e.g., matches, or substantially matches, the circumference of an arcuate shaped stent such as described in U.S. Pat. No. 11,672,702 so as not to place strain energy into the stent that would cause it to change its shape, e.g., to spring back to its original shape, as it exits the lumen at the distal tip 106 of the cannula 100 which would make controlled delivery of the stent into Schlemm's canal more difficult.

The circumference of proximal curved segment 110 enables the distal tip 106 located at the distal end of distal curved segment 105 of the cannula 100 to be on the centerline of the longitudinal axis of straight segment 115 located at the proximal portion of the cannula 100. In one embodiment, the straight segment has a length L4. In one embodiment, the length L1 comprises the lengths L2, L3 and L4.

In one embodiment, the circumference value for distal curved segment 105 directly corresponds to the shape and flexibility (mechanical-elastic modulus) of the medical device being delivered through the lumen of the cannula 100. A straight, stiff, medical device would benefit from a larger circumference for distal curved segment 105, whereas a small curved stiff medical device would benefit from a smaller circumference, for example, a circumference that is substantially the same, or the same, as the circumference for distal curved segment 105. A flexible medical device would make the circumference of distal curved segment 105 more arbitrary as the flexible medical device would exit the lumen of the cannula 100 at distal tip 106 in the direction, or substantially in the direction, of the tangent of the circumference of Schlemm's canal which enables easier passage of the medical device through Schlemm's canal.

The circumference of proximal curved segment 110 brings the distal tip 106 of the cannula 100 (located at the distal end of distal curved segment 105) back to the centerline of the straight segment 115 located at the proximal portion of the cannula 100. The benefit of having the distal tip 106 located at the centerline of the straight proximal segment 115 of the cannula 100 is that doing so enables the distal tip 106 of the cannula 100 to be on the centerline of the straight segment 115 of the cannula 100, which, in turn, is aligned with the centerline of an inserter handle (not shown) and enables a surgeon to anticipate the angular location (zero angle) and offset from the centerline from the handle (zero offset) of the distal tip 106 of the cannula 100 that is aligned with the centerline of the inserter handle.

In one embodiment, the length L2 of distal curved segment 105 illustrated in FIG. 2 corresponds directly to the arc length and angle of distal curved segment 105. In one embodiment, the length L2 is chosen to enable the lowest possible induced stress/strain placed on the medical device that is delivered from the distal tip 106 of cannula 100 into the anatomy of the eye, i.e., Schlemm's canal. Range of values for L2 are from arbitrarily very small, say 0.1 mm, to slightly more than the diameter of the white-to-white (iris 160) of 12 mm.

In one embodiment, the length L3 of proximal curved segment 110 illustrated in FIG. 2 corresponds to the arc length of proximal curved segment 110. L3 is determined by the arc length of proximal curved segment 110 that is needed to bring the distal tip 106 of the cannula 100 to the centerline of the straight segment 115 at the base part, i.e., proximal portion, of the cannula 100. Range of values for L3 are from arbitrarily very small, for example, 0.1 mm, to slightly more than the diameter of the white-to-white (iris 160) of 12 mm.

The lengths of L2 and L3 may be chosen so that a medical device, such as the stent described in U.S. Pat. No. 11,672,702 with an arc radius of, for example, 5.5 mm can be delivered through the proximal curved segment 100 with no or minimal induced strain on the stent so that it will not change shape, or spring back into another position or shape when it exits the distal tip 106 of cannula 100 due to its passage through the lumen of the cannula 100. Presently the stent described in U.S. Pat. No. 11,672,702 is a one-size-fits-all stent since there is very little variability in the world's adult population white-to-white (iris 160) diameter so there is only one cannula geometry corresponding to one stent geometry.

In one embodiment, the arc length of proximal curved segment 110 is maximized or optimized to minimize the length of the medical device, i.e., a stent residing in distal curved segment 105 which may be under strain in proximal curved segment 110 and correspondingly have greater contact force with the inside wall of the cannula 100 and increase the frictional force between the stent and the cannula 100 requiring greater axial force on the proximal end of the stent to push it out of the lumen at the distal tip 106 of the cannula 100, which would make delivery of the stent into Schlemm's canal more difficult.

It is appreciated that proximal curved segment 110 in another embodiment curves to the right and distal curved segment 105 curves to the left. More generally, the curved segments curve in respectively opposite x directions within the same x-y plane. The general configuration aligns the distal tip 106 of the cannula 100 with the centerline of the proximal straight segment 115 of the cannula 100 which itself is aligned to the centerline of the inserter handle (not shown). This enables the user that is gripping the inserter handle to anticipate where the distal tip 106 of the cannula 100 is with respect to the distal tip's angular offset from the centerline of the handle (which is zero angle), and the offset from the axis of the inserter handle (which is zero offset). Having this direct correspondence between the distal tip 106 to the centerline of the inserter handle makes it easier to anticipate the position of the distal tip 106 while the cannula is positioned in the eye.

It is appreciated that the distal and proximal curve segments 105, 110 could curve in different planes (e.g., different x and z directions). For example, distal curved segment 105 and proximal curved segment 110 may curve in different planes rotated relative to one another.

In one embodiment, the straight segment 115 depicted in FIGS. 1 and 2 and distal and proximal curved segments 105, 110 are configured such that the distal tip 106 located at the distal end of distal curved segment 105 is in axial alignment with the straight segment 115. In other words, the distal tip 106 is positioned at approximately the same location on the x-axis as the straight segment 115, in an x-y plane. The benefit of having the distal tip 106 located at the centerline of the proximal straight segment 115 of the cannula 100 is that doing so enables the distal tip 106 of the cannula 100 to be on the centerline of the longitudinal axis of the proximal straight segment 115 of the cannula 100 which, in turn, is aligned with the centerline of the inserter handle. This configuration enables a surgeon to anticipate the angular location (zero angle) and offset of the distal tip from the centerline from the inserter handle (zero offset).

It is appreciated that the distal tip 106 may be slightly to the left or right of the same location on the x-axis as the straight segment 115. This distance to the left or right of the same location on the x-axis as the straight segment 115 may range from zero to half the diameter of the white-to-white (iris 160) of the eye (max 12 mm) which is 6 mm.

Although embodiments of the invention have been described in considerable detail, other embodiments are possible. It will be apparent to one with skill in the art that embodiments of the device may be provided using some or all the aforementioned features and components without departing from the spirit and scope of the invention. It will also be apparent to the skilled artisan that the embodiments described above are specific examples of a single broader device which may have greater scope than any of the singular descriptions taught. There may be many alterations made in the descriptions without departing from the spirit and scope of the invention. Therefore, the spirit and scope of the appended claims should not be limited to the description of the embodiments contained therein.

1. A multiple curved cannula, comprising:
a proximal curved segment having a proximal end and a distal end;
a distal curved segment having a proximal end and a distal end, wherein the proximal end of the distal curved segment is adjacent the distal end of the proximal curved segment to form the multiple curved cannula.

2. The multiple curved cannula of claim 1, further comprising a proximal straight segment having a proximal end and a distal end, wherein the distal end of the proximal straight segment is adjacent the proximal end of the proximal curved segment.

3. The multiple curved cannula of claim 2, further comprising an inserter handle coupled to the proximal end of the proximal straight segment.

4. The multiple curved cannula of claim 3, wherein the inserter handle coupled to the proximal end of the proximal straight segment comprises the inserter handle adjoined to the proximal end of the proximal straight segment.

5. The multiple curved cannula of claim 1, further comprising:
a lumen; and
wherein a medical device can pass through the lumen and exit the multiple curved cannula via the distal end of the distal curved segment.

6. The multiple curved cannula of claim 5, wherein the medical device can enter through the proximal end of the proximal curved segment and pass through the lumen to the distal end of the distal curved segment.

7. The multiple curved cannula of claim 1, wherein a location of a first center of a first radius of a first curvature for the distal curved segment is different than a location of a second center of a second radius of a second curvature for the proximal curved segment.

8. The multiple curved cannula of claim 1, wherein a length of the distal curved segment ranges from 0.1 mm to 12 mm and/or a length of the proximal curved segment ranges from 0.1 mm to 12 mm.

9. The multiple curved cannula of claim 1, wherein a length of the distal curved segment is less than a length of the proximal curved segment.

10. The multiple curved cannula of claim 2, wherein a length of the proximal straight segment is less than a length of the distal curved segment and less than a length of the proximal curved segment.

11. The multiple curved cannula of claim 1, wherein the distal curved segment defines a first arc of a first circle having a first circumference and the proximal curved segment defines a second arc of a second circle having a second circumference that is greater than the first circumference.

12. The multiple curved cannula of claim 1, wherein the first circumference approximates a circumference of Schlemm's canal in a mammalian eye.

13. The multiple curved cannula of claim 1, wherein the distal curved segment comprises a circumference that substantially corresponds to a circumference of a medical device that can be inserted in a lumen of the multiple curved cannula.

14. The multiple curved cannula of claim 2, wherein the distal end of the distal curved segment is in substantial axial alignment with a longitudinal axis of the proximal straight segment.

15. The multiple curved cannula of claim 14, wherein the longitudinal axis of the proximal straight segment is aligned with a longitudinal axis of an inserter handle coupled to the proximal end of the proximal straight segment.

16. The multiple curved cannula of claim 1, wherein the proximal curved segment and the distal curved segment are aligned in a x-y plane, wherein the proximal curved segment curves to the left in the x-y plane and the distal curved segment curves to the right in the x-y plane.

17. The multiple curved cannula of claim 1, wherein the proximal curved segment is aligned with a first plane and the distal curved segment is aligned with a second plane that is rotated relative to the first plane.
